(19) United States
(12) Patent Application Publication
(10) Pub. No.: US 2015/0315655 A1
(43) Pub. Date: Nov. 5, 2015

(54) PROSTATE CANCER TESTING AND DIAGNOSIS METHOD

(71) Applicant: Tricia Vicedomine, Averill Park, NY (US)

(72) Inventor: Tricia Vicedomine, Averill Park, NY (US)

(21) Appl. No.: 14/530,882

(22) Filed: Nov. 3, 2014

Related U.S. Application Data
(60) Provisional application No. 61/873,722, filed on Sep. 4, 2013.

(51) Int. Cl. C12Q 1/68

(52) U.S. Cl. CPC C12Q 1/6886; C12Q 2600/154; C12Q 2600/158; C12Q 2600/16

(57) ABSTRACT

A molecular assay of prostate cancer testing and diagnosis method is provided. The tests of the present invention include a broad spectrum analysis of testing molecular values for prostate cancer management. The tests of the present invention may include a three part series of testing which includes distinguishing benign prostate hyperplasia from a potential prostate cancer diagnosis. The present invention further examines recurrent values for prostate cancer patients and in addition measures values for metastatic prostate cancer.

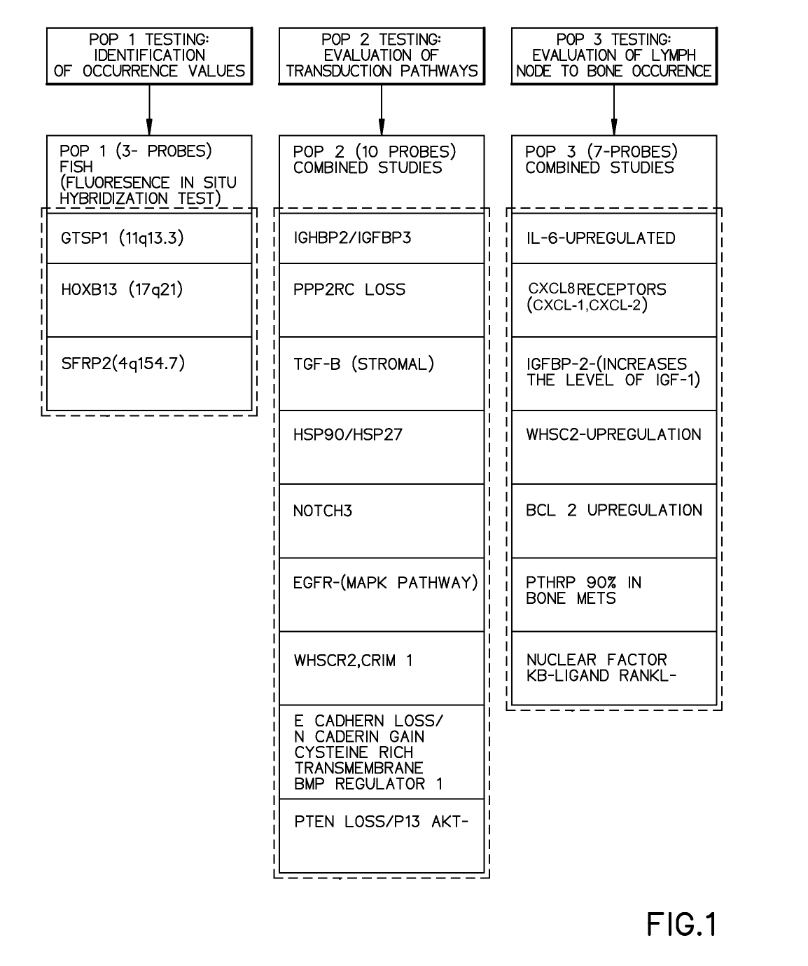

### CROSS-REFERENCE TO RELATED APPLICATION

This application claims the benefit of priority of U.S. provisional application No. 61/873,722, filed Sep. 4, 2013, the contents of which are herein incorporated by reference.

### BACKGROUND OF THE INVENTION

The present invention relates to prostate cancer and, more particularly, to a prostate cancer testing and molecular profiling of the disease.

Prostate cancer, also known as carcinoma of the prostate, is the development of cancer in the prostate, a gland in the male reproductive system. Most prostate cancers are slow growing; however, some grow relatively fast. The cancer cells may spread from the prostate to other parts of the body, particularly the bones and lymph nodes.

Currently, technology is permitting us to look at molecular expressions of tumors, but most physicians are not trained in advanced molecular technologies. With accelerating development of personalized cancer treatments matched to a patient's expressive values, frontline physicians increasingly need help to maneuver through the complex genomic landscape to find the most effective, individualized therapy.

As can be seen, there is a need for improved ancillary molecular testing profiling the screening for prostate cancer patients.

### SUMMARY OF THE INVENTION

In one aspect of the present invention, a method of testing a level of probability for a patient to have greater predictive values of prostate cancer comprises the steps of: determining whether the patient has a hypermethylation of Glutathione S Transferase; determining whether the patient has a methylation of secreted frizzle-related protein 2; and determining whether the patient has a homeobox protein mutation (HOXB13 mutation).

In another aspect of the present invention, a method of evaluating expressive recurrent values in transcription/transduction pathways of a patient with prostate cancer comprises: measuring the level of insulin like growth factor binding protein 2 and 3 within the patient; determining if there is PPP2RC loss within the patient; determining if there is overexpression of transforming growth factor beta within the patient; determining if there is overexpression of heat shock protein 90 and 27 within the patient; determining if there is overexpression of Neurogenic locus notch homolog protein 3 within the patient; determining if there is epidermal growth factor receptor inhibition within the patient; determining if there is upregulation of WHSCR2 and Cysteine-rich motor neuron 1 protein within the patient; determining if there is an E-cadherin loss or N-Cadherin gain within the patient; and determining if there is a phosphatase and tensin homolog loss within the patient.

In another aspect of the present invention, a method of testing a level of probability for a patient with prostate cancer to transition into metastasis of lymph node to bone comprises: determining if there is an upregulation of Interleukin 6 within the patient; determining if there is an overexpression of CXCL8 receptors (CXCL-1, CXCL-2)within the patient; testing for insulin-like growth factor-binding protein 2 within the patient; determining if there is an upregulation of WHSCR2 within the patient; determining if there is an overexpression of BCL-2 within the patient; determining a level of parathyroid hormone-related protein within the patient; and testing for nuclear factor KB-ligand RANKL within the patient.

These and other features, aspects and advantages of the present invention will become better understood with reference to the following drawings, description and claims.

### BRIEF DESCRIPTION OF THE DRAWINGS

The FIGURE is a schematic view of the three phases of testing of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a Prostate Oncogenic Predictives (POP) test. The POP tests are a broad spectrum analysis serving as an ancillary molecular amenity for profiling a patient's of prostate cancer. The POP tests may include a three part series of testing and may solve differential issues that distinguish benign prostate hyperplasia from more concerning issues. The present invention further examines recurrent values for prostate cancer patients and measures values for metastatic prostate cancer.

Referring to the FIGURE, the present invention may include three POP tests, POP 1, 2, and 3. POP 1 primarily differentiates a benign prostate hyperplasia (BHP) with more advanced concerns. POP 2 looks at known expressive values that bypass androgen deprivation therapies. POP 3 looks at protocols assessed for lymph node and bone metastasis.

The POP 1 test of the present invention may test for a patient's hereditary prostate cancer, as well as differentiation potentials of BHP and high grades Prostate Intraepithelial Neoplasia (PINS). The POP 1 test may test for hypermethylated Glutathione S Transferase (GSTP1), methylation frequency of secreted frizzled-related protein 2 (SFRP2), and the homeobox protein (HOXB13) mutation. If tests show positive values for at least one of the three, the patient is likely to have clinically correlated predictive values that suggest further concern and advanced monitoring for prostate cancer.

GSTP1 is a protein that typically displays hypermethylation if the patient has prostate cancer. The protein rarely displays hypermethylation in normal prostates as well as during hyperplasic prostate epithelium. GSTP1 is the most consistent hypermethylated marker in prostate cancer patients.

Gene expression and epigenetic discovery screen reveal methylation of SFRP2 in prostate cancer patients. SFRP2 methylation was detected at significantly lower frequencies in high-grade prostatic intraepithelial neoplasia (HGPIN; 30%, (6/20), p=0.0096), tumor adjacent benign areas (8.82%, (7/69), p<0.0001) and BPH (11.43% (4/35), p<0.0001). The quantitative level of SFRP2 methylation (normalized index of methylation) was also significantly higher in tumors than in the other samples (HGPIN=7.45, HB=0.47, and BPH=0.12). SFRP2 hypermethylation is a common event in prostate cancer. SFRP2 methylation in combination with other epigenetic markers may be a useful biomarker of prostate cancer.

The last of the POP 1 test is the HOXB13 mutation test. G84E mutation frequency was 0.99% and 0.24% in positive and negative biopsy subjects respectively. In positive biopsy subjects, the frequency was significantly higher in subjects with a positive family history than those without (4.31% versus 0.34%). The carrier rate of the G84E mutation was increased by a factor of approximately 20 in 5083 unrelated subjects of European descent who had prostate cancer, with the mutation found in 72 subjects (1.4%), as compared with 1 in 1401 control subjects (0.1%). The mutation was significantly more common in men with early-onset, familial prostate cancer (3.1%) than in those with late-onset, nonfamilial prostate cancer (0.6%) (P=2.0×10⁻⁶). Patients who possessed the variant were more likely to have a family history of prostate cancer (46.0% vs. 35.4%). G84E carriers were also more likely diagnosed at a younger age compared to non-carriers (55.2 years vs. 58.1 years). The HOXB13 mutation substantially increases risk of early onset, familial prostate cancer in European-American men.

The POP 2 test of the preset invention monitors, evaluates, and measures known components of correlations with patient prognosis in recurrent values of malignant human prostate tumors. In particular, the POP 2 test is an evaluation of known values that promote aggressive cancer outcomes and bypass androgen deprivation therapies and the transduction pathways of the patient.

The first test of the POP 2 test is to measure levels of insulin like growth factor binding protein 2 and 3 (IGH-BP-2-3). Insulin-like growth factor-binding protein-2 promotes prostate cancer cell growth via IGF-dependent or -independent mechanisms and reduces the efficacy of docetaxel. The IGFBP-2 has a key role in the growth of prostate cancer cells, and silencing IGFBP-2 expression reduces the resistance of these cells to docetaxel. Therefore, targeting IGFBP-2 may increase the efficacy of docetaxel. IGF− (Insulin Like Growth factor) is an upstream effector on AKT signaling, and an IGF upregulation (which activates AKT) suggests an inter relationship. IGF-1 (stromal) PSA cleaves IGFBF3 causing and increase in IGF-1. The next test of the POP 2 test is to determine if there is PPP2RC loss. PPP2RC loss promotes castrate resistant prostate cancer growth and is associated with increased prostate cancer specific mortality. The identification of pharmalogical inhibitors of PPP2R2C complexes or the growth pathways activated by PPP2Rc loss may prove useful as a therapeutic value in men with CRPC.

The next test of the POP 2 test may include the determination of the over expression of transforming growth factor beta (TGF-B). The over expression may be caused by the stromal micro environment produced by CD4 +T cells/inhibition and down regulation of cyclin D. TGF-B is a multifunctional cytokine with an established role as a pro-metastatic agent in advanced cancers, and its expression has been negatively correlated with patient prognosis in malignant human prostate tumors. TGF-B activated ligands compose a very diverse cysteine rich domain, along with down regulation of cyclin D. These effects are indirectly through stromal cells. Higher levels of TGF-B in the sera of prostate cancer patients are associated with bone metastasis. In prostate cancer cells, TGF-B1 stimulates the expression and activity of UPA (Urokinase type plasmogen actovator system), PA1, MMP-9, resulting in a net increment of pericellular plasminogen activation and finally increased tumor cell metastasis and invasion.

The next test of the POP 2 test is to determine elevated expression of Heat Shock Proteins (HSP) 90 and HSP 27. HSP 90 is another multifaceted molecular chaperone implicated in the progression of prostate cancer by the induction of several upstream signaling pathways, which promote aberrant androgen receptor activation and stabilize the androgen receptor protein. HSP regulates epithelial mesenchymal transitions. HSP 27 increases expression of Vimentin gain, fibronectin gain, n-cadherin gain, and E-cadherin loss. HSP 27 regulates epithelial to mesenchymal transitions (EMT), and is a critical regulator of Interleukin 6 (IL-6) dependent and independent EMT. Elevated expression of HSP 27 and HSP 90 is associated with a increased chance of prognosis of metastatic prostate cancer.

The next test of the POP 2 test includes determining if there is elevated expression of Neurogenic locus notch homolog protein 3 (NOTCH 3). Stromal vimentin and Notch 3 activity is increased in prostate cancers, suggesting a pro-metastatic function of Notch signaling during prostate cancer progression.

The next test of the POP 2 test includes determining if there is epidermal growth factor receptor (EGFR) inhibition. EGFR-inhibition (MAP K Pathway/Later stages of the disease) was found to have multiple roles in prostatic tumorigenesis.

The next test of the POP 2 test includes determining if there is upregulation of WHSCR2 and Cysteine-rich motor neuron 1 protein (CRM 1). WHSC2 is a nuclear protein expressed in various tissues and in different cell lines. CRIM1 is a glycosylated, Type I transmembrane protein and it has been demonstrated that the extracellular CRR-containing domain can also be secreted, presumably via processing at the membrane. Crim1 expression at sites is consistent with an interaction with bone morphogenetic proteins (BMPs).

The next test of the POP 2 test includes determining if there is an E-cadherin loss /N-Cadherin gain. Cadherins are a class of type-1 transmembrane glycoproteins, of which E-cadherin and N-cadherin are the best characterized in prostate cancers. E-cadherin is an important tumor suppressor gene. Loss of E-cadherin expression disrupts cell-cell junctions and consequently promotes cell migration, leading to tumor metastasis. Multiple studies reported that loss of E-cadherin expression enhances the progression from nonmetastatic to metastatic carcinoma. In primary prostate cancers, reduced E-cadherin expression has been correlated with increased tumor grade, bone metastasis, and poor prognosis. In contrast, increased levels of N-cadherin and cadherin-11 are associated with poorly differentiated and metastatic prostate cancers. In more aggressive prostate cancer specimens, N-cadherin expression is increased while E-cadherin is reduced. Androgen deprivation therapy leads to elevated expression of E-cadherin in prostate cancer, suggesting that androgens repress E-cadherin expression.

The next test of the POP 2 test includes determining if there is a phosphatase and tensin homolog (PTEN) loss. PTEN loss upregulates P13/AKT/mTor, which leads to AKT hyperactivation. Further, PTEN is haplo-insufficient, linked to progression, and is correlated to more advanced PCa resistance to sunitinib which correlates to loss of Pten expression. The lipid and protein phosphatase PTEN is a key negative regulator of Akt activity. Aberrant expression of PI3K and AKT1 genes or loss of PTEN tumor suppression gene leads to downstream upregulation of mTOR and tumorigenesis.

The POP 3 test includes measured evaluated factors for prostate cancer patients that have shown metastasis to lymph node and bone. The POP 3 test may include, but is not limited to: testing for upregulated IL-6; testing for the overexpression of CXCL-8 receptors (CXCL-1.CXCL2); testing for Insulin-like growth factor-binding protein 2 (IGFBP-2) which increases the level of IGF-1; testing for upregulated WHSC2; testing for upregulated BCL-2; testing for upregulated BCL 2; testing the PTHRP level, which may be 90% in bone metastic patients; and testing for Nuclear factor KB-ligand RANKL.

Cytokines are a family of immunoregulatory peptide growth factors. They are produced mainly by immune cells after immune challenge (infection/inflammation). Various cells of nonimmune cell origin such as epithelial, muscle, endothelial, fibroblast, mesenchymal, and also sperm cells are capable of producing cytokines. Cytokines are pleiotropic factors with autocrine and paracrine effects and may therefore play an important role in the testicular microenvironment. The main proinflammatory cytokines are tumor necrosis factor-α (TNF-α), interleukin (IL)-1, and IL-6. The first cytokine produced after immunologic challenge is TNF-α, which induces the production of IL-1 followed by IL-6. IL-6 is a 21-28 kDa glycoprotein produced by T and B cells, monocytes, macrophages, fibroblasts, endothelial cells, and tumor cells.

In prostate cancer CXCL8 over-expression induced the expression of MMP-9 that in turn increased tumor cell invasiveness and metastatic potential.

Inflammatory cytokines such as CXCL1 and CXCL8 can enhance tumor cell proliferation and have effects on angiogenesis.

The insulin like growth factor (IGF) pathway, which includes IGF receptor-1 (IGF-1 R) and its ligands, IGF-I and IGF-II, not only plays a major role in growth, development, and maintenance of homeostasis in normal cells, but also the proliferation of cancers cells, including prostate cancer cells. Higher levels of circulating IGF-I correlates with increased risk of developing prostate cancer, as well as metastatic disease. Blockade of IGF-IR in combination with chemotherapy leads to chemosensitization in androgen-independent prostate cancer cell lines and improved docetaxel antitumor activity in animal models. Similar positive associations with IGF-I levels have also been found for prostate cancer. Androgen action is dependent on IGF-1 signaling. IGF-1 interacts with IGF-1R to induce PI3K/AKT activation, which then regulates the activities of mTOR and FOXO1.

Overexpression of BCL-2 is observed in a significant number of men with castrate resistant prostate cancer (CRPC). It plays a key role in the onset of castration refractoriness and contributes to the resistance to radiation and docetaxel therapy. Inhibition of BCL-2 expression leads to increased apoptosis, as well as diminished proliferation and angiogenesis.

Parathyroid hormone-related protein (PTHrP) possesses a variety of physiological and developmental functions and is also known to facilitate the progression of many common cancers, notably their skeletal invasion, primarily by increasing bone resorption. PTHrP may work through epithelial-to-mesenchymal transition to promote an aggressive and metastatic phenotype in prostate cancer, a pathway of importance in cancer stem cells. Quantitatively measuring PTHrP, as part of POP 3 testing, as well as developing ways to specifically target PTHrP signaling may lead to more effective therapies for prostate cancer.

There is a strong association between the nuclear localization of nuclear factor-kappa B (NF-jB) p65, and prostate cancer aggressiveness and biochemical recurrence. The majority of patients with mCRPC develop osteoblastic bone metastasis accompanied by simultaneous bone destruction, due to increased osteoclastic activity. The receptor activator of nuclear factor-kB ligand (RANKL) is critical for the formation, function, and survival of osteoclasts. In preclinical models of prostate cancer, inhibition of osteoclasts leads to improvement of sclerotic changes in the bones. In a phase III trial, zoledronic acid, an inhibitor of osteoclasts, significantly decreased the incidence of skeletal related events (SRE) and increased the median time to the first SRE, over placebo. Denosumab is a monoclonal antibody against RANKL. The Nuclear factor kappa B (NF-jB) transcription factor family is composed of the p65, RelB, c-Rel, p50/p105 and p52/p100 subunits that function as homo- or hetero-dimers. Denosumab is a monoclonal antibody against RANKL. Compared to zoledronic acid, denosumab improved median time to first SRE in a phase III trial of men with CRPC. Nuclear NF-KB expression in primary prostate cancers is highly predictive for pelvic lymph node metastases. The coordinated regulation of cancer cell invasion and metastasis by the feed forward mechanism involving RANKL, c-Met, transcription factors, and VEGF-neuropilin could offer new therapeutic opportunities to target prostate cancer bone and soft tissue metastases.

The testing performed in the POP 1, 2, and 3 test may include quantitative analysis methodologies. The quantitative analysis methodologies may include, but are not limited to, PCR/Microarrays, Immuno-histochemistry, Nanotechnology, Flourescent InSitu Hybridization (FISH), and Infrared based Protein Quantization using direct detect spectrometry.

It should be understood, of course, that the foregoing relates to exemplary embodiments of the invention and that modifications may be made without departing from the spirit and scope of the invention as set forth in the following claims.

What is claimed is:

1. A method of testing a level of probability for a patient to develop prostate cancer comprising the steps of:
determining whether the patient has a hypermethylation of Glutathione S Transferase;
determining whether the patient has a methylation of secreted frizzle-related protein 2; and
determining whether the patient has a homeobox protein mutation.

2. The method of claim 1, wherein the patient has an enlarged prostate prior to the testing.

3. The method of claim 2, wherein testing positive for at least one of the hypermethylation of Glutathione S Transferase, the methylation of secreted frizzle-related protein 2, and the homeobox protein mutation increases the level of probability for the patient to have prostate cancer.

4. The method of claim 2, wherein testing negative for the hypermethylation of Glutathione S Transferase, the methylation of secreted frizzle-related protein 2, and the homeobox protein mutation increases the level of probability for the patient to have benign prostatic hyperplasia.

5. A method of evaluating the androgen deprivation transduction pathways of a patient with prostate cancer comprising:
measuring the level of insulin like growth factor binding protein 2 and 3 within the patient;
determining if there is PPP2RC loss within the patient;
determining if there is overexpression of transforming growth factor beta within the patient;
determining if there is overexpression of heat shock protein 90 and 27 within the patient;
determining if there is overexpression of Neurogenic locus notch homolog protein 3 within the patient;
determining if there is epidermal growth factor receptor inhibition within the patient;
determining if there is upregulation of WHSCR2 and Cysteine-rich motor neuron 1 protein within the patient;
determining if there is an E-cadherin loss or N-Cadherin gain within the patient; and
determining if there is a phosphatase and tensin homolog loss within the patient.

6. A method of testing a level of probability for a patient with prostate cancer to acquire metastasis of lymph node to bone comprising:
determining if there is an upregulation of Interleukin 6 within the patient;
determining if there is an overexpression of CXCL8 receptors within the patient;
testing for insulin-like growth factor-binding protein 2 within the patient;
determining if there is an upregulation of WHSCR2 within the patient;
determining if there is an overexpression of B-cell lymphoma 2 within the patient;
determining a level of parathyroid hormone-related protein within the patient; and
determining for nuclear factor KB-ligand RANKL within the patient.
